(19) United States
(12) Patent Application Publication
(10) Pub. No.: US 2025/0161394 A1
(43) Pub. Date: May 22, 2025

(54) Compositions Comprising Kavalactones

(71) Applicant: Natural Extraction Systems, LLC, Boulder, CO (US)

(72) Inventor: C. Russell Thomas, Boulder, CO (US)

(21) Appl. No.: 18/951,457

(22) Filed: Nov. 18, 2024

Related U.S. Application Data
(60) Provisional application No. 63/600,652, filed on Nov. 18, 2023.

(51) Int. Cl. A61K 36/67; A61K 9/00; A61K 47/46

(52) U.S. Cl. CPC A61K 36/67; A61K 9/006; A61K 47/46

(57) ABSTRACT

Various aspects of this disclosure relate to compositions comprising one or more kavalactones, one or more aromatic compounds and/or cinnamaldehyde, and a solvent and/or cosolvent that is an alcohol such as ethanol, propylene glycol, or glycerol. In some embodiments, the composition is an oral spray. Such compositions are suitable to administer a psychoactive dose of the one or more kavalactones having improved pharmacokinetics, improved bioavailability, attenuated side effects, and/or improved flavor profiles relative to conventional kavalactone formulations.

### CROSS-REFERENCE TO RELATED APPLICATIONS

This patent application claims priority to U.S. Provisional Patent Application No. 63/600,652, filed Nov. 18, 2023, which is hereby incorporated by reference in its entirety.

### BACKGROUND

Kava (Piper methysticum) contains psychoactive compounds and may confer certain health benefits. Kava supplements can help regulate anxiety, mood, and/or sleep, and they may also display anti-inflammatory, anti-depressant, and/or neuroprotective effects.

For millennia, kava has been consumed as a traditional beverage throughout Polynesia. Kava root is harvested, processed into a paste, and brewed into a tea that provides gentle mood-enhancing and anxiolytic effects. While kava is consumed throughout Polynesia, its unfavorable flavor, poor bioavailability, slow pharmacokinetics, and side effects including stomach distress limit broader adoption in Western society.

Kava displays a bitter, earthy, and sulfurous flavor profile that is difficult to mask. The name kava literally means bitter in the Tongan and Marquesan languages. The onset of action is also relatively long with effects generally noticeable only after 45 minutes or more.

Improved formulations that display the favorable pharmacological properties of kava without unfavorable flavors, pharmacokinetics, bioavailability, and side effects are desirable.

### SUMMARY

Various aspects of this disclosure relate to compositions comprising kavalactones that display improved properties relative to prior art formulations including improved flavor profiles, improved pharmacokinetics, improved bioavailability, and attenuated side effects.

Various aspects of this disclosure relate to the discovery that compositions comprising kavalactones dissolved in a liquid formulation with one or more alcohols as a solvent and/or cosolvent display improved pharmacokinetics relative to prior-art formulations. Without limiting this disclosure or any patent claim that matures from this disclosure, compositions comprising kavalactones that are dissolved in a liquid formulation with one or more alcohols as a solvent and/or cosolvent display improved pharmacokinetics because such compositions allow absorption of the kavalactones by the epithelial lining of the gastrointestinal tract between the lips and the stomach (i.e., including the lining of the mouth and esophagus and excluding the lining of the stomach). In contrast, prior-art formulations favor absorption in one or more of the stomach, small intestine, and large intestine, which delays the onset of psychoactive effects. Suitable alcohols include, without limitation, ethanol, propylene glycol, and glycerol. Improved pharmacokinetics may be identified, for example, by comparing the Tmax of compositions of this disclosure with the Tmax of prior-art formulations.

Various aspects of this disclosure relate to the discovery that compositions comprising kavalactones dissolved in a liquid formulation with one or more alcohols as a solvent and/or cosolvent display improved bioavailability relative to prior-art formulations. Without limiting this disclosure or any patent claim that matures from this disclosure, compositions comprising kavalactones that are dissolved in a liquid formulation with one or more alcohols as a solvent and/or cosolvent display improved bioavailability because such compositions allow absorption of the kavalactones by the epithelial lining of the gastrointestinal tract between the lips and the stomach. In contrast, prior-art formulations favor absorption in one or more of the stomach, small intestine, and large intestine, which subjects the kavalactones to acidic conditions and/or alkaline conditions and/or the gut microbiome and/or first-pass metabolism that can chemically degrade the kavalactones and reduce bioavailability. Suitable alcohols include, without limitation, ethanol, propylene glycol, and glycerol. Improved bioavailability may be identified, for example, by comparing plasma concentrations of kavalactones following the administration of compositions of this disclosure with plasma concentrations following the administration of prior-art formulations.

Various aspects of this disclosure relate to the discovery that compositions comprising kavalactones dissolved in a liquid formulation with one or more alcohols as a solvent and/or cosolvent display attenuated side effects relative to prior-art formulations. Without limiting this disclosure or any patent claim that matures from this disclosure, compositions comprising kavalactones that are dissolved in a liquid formulation with one or more alcohols as a solvent and/or cosolvent display attenuated side effects because such compositions allow absorption of the kavalactones by the epithelial lining of the gastrointestinal tract between the lips and the stomach, which minimizes gastrointestinal distress and/or which minimizes first-pass metabolism thereby minimizing liver burden. Suitable alcohols include, without limitation, ethanol, propylene glycol, and glycerol.

Various aspects of this disclosure relate to the discovery that compositions comprising kavalactones formulated as an oral spray display improved pharmacokinetics relative to prior-art formulations. Without limiting this disclosure or any patent claim that matures from this disclosure, compositions comprising kavalactones formulated as an oral spray display improved pharmacokinetics because such compositions favor absorption of the kavalactones by the epithelial lining of the gastrointestinal tract between the lips and the stomach. In contrast, prior-art formulations favor absorption in one or more of the stomach, small intestine, and large intestine, which delays onset of action. Absorption of the kavalactones by the epithelial lining of the gastrointestinal tract between the lips and the stomach is favored in spray formats relative to liquid formats, for example, because sprays present greater surface area relative to liquids.

Various aspects of this disclosure relate to the discovery that compositions comprising kavalactones formulated as an oral spray display improved bioavailability relative to prior-art formulations. Without limiting this disclosure or any patent claim that matures from this disclosure, compositions comprising kavalactones formulated as an oral spray display improved pharmacokinetics display improved bioavailability because such compositions favor absorption of the kavalactones by the epithelial lining of the gastrointestinal tract between the lips and the stomach. In contrast, prior-art formulations favor absorption in one or more of the stomach, small intestine, and large intestine, which subjects the kavalactones to acidic conditions and/or alkaline conditions and/or the gut microbiome and/or first-pass metabolism that can chemically degrade the kavalactones and reduce bioavailability.

Various aspects of this disclosure relate to the discovery that compositions comprising kavalactones formulated as an oral spray display attenuated side effects relative to prior-art formulations. Without limiting this disclosure or any patent claim that matures from this disclosure, compositions comprising kavalactones formulated as an oral spray display attenuated side effects because such compositions favor absorption of the kavalactones by the epithelial lining of the gastrointestinal tract between the lips and the stomach, which minimizes gastrointestinal distress and/or which minimizes first-pass metabolism thereby minimizing liver burden.

Various aspects of this disclosure relate to the discovery that compositions comprising kavalactones formulated with other aromatic compounds display improved pharmacokinetics relative to prior-art formulations. Without limiting this disclosure or any patent claim that matures from this disclosure, compositions comprising kavalactones formulated with other aromatic compounds allow for non-covalent interactions such as pi-stacking interactions between the kavalactones and the aromatic compounds, which inhibits aggregation of the kavalactones and/or allows dispersal of the kavalactones in the composition, which inhibition of aggregation and/or dispersal increases the probability that the kavalactones will contact and be absorbed by the epithelial lining of the gastrointestinal tract between the lips and the stomach and therefore improves pharmacokinetics (e.g., as described above). Suitable aromatics include, without limitation, anthocyanins, glycosides of anthocyanins, flavonoids, and glycosides of flavonoids as well as other aromatic compounds described in the detailed description that follows. An anthocyanin carries a net positive electrostatic charge, for example, and non-covalent interactions between anthocyanins and kavalactones can therefore inhibit aggregation and/or disperse kavalactones by electrostatic repulsion. Glycosides of anthocyanins, flavonoids, and other aromatic compounds also have steric bulk as a result of covalently-bound sugars, which steric bulk can inhibit aggregation and/or disperse kavalactones in liquids.

Various aspects of this disclosure relate to the discovery that compositions comprising kavalactones formulated with other aromatic compounds display improved bioavailability relative to prior-art formulations. Without limiting this disclosure or any patent claim that matures from this disclosure, compositions comprising kavalactones formulated with other aromatic compounds allow for non-covalent interactions such as pi-stacking interactions between the kavalactones and the aromatic compounds, which inhibits aggregation of the kavalactones and/or allows dispersal of the kavalactones in a composition, which inhibition of aggregation and/or dispersal increases the probability that the kavalactones will contact and be absorbed by the epithelial lining of the gastrointestinal tract between the lips and the stomach and therefore improves bioavailability (e.g., as described above). Suitable aromatics include, without limitation, anthocyanins, glycosides of anthocyanins, flavonoids, and glycosides of flavonoids as well as other aromatic compounds described in the detailed description that follows.

Various aspects of this disclosure relate to the discovery that compositions comprising kavalactones formulated with other aromatic compounds display attenuated side effects relative to prior-art formulations. Without limiting this disclosure or any patent claim that matures from this disclosure, compositions comprising kavalactones formulated with other aromatic compounds allow for non-covalent interactions such as pi-stacking interactions between the kavalactones and the aromatic compounds, which inhibits aggregation of the kavalactones and/or allows dispersal of the kavalactones in a composition, which inhibition of aggregation and/or dispersal increases the probability that the kavalactones will contact and be absorbed by the epithelial lining of the gastrointestinal tract between the lips and the stomach and therefore attenuates side effects (e.g., as described above). Suitable aromatics include, without limitation, anthocyanins, glycosides of anthocyanins, flavonoids, and glycosides of flavonoids as well as other aromatic compounds described in the detailed description that follows.

Various aspects of this disclosure relate to the discovery that compositions comprising kavalactones formulated with other aromatic compounds display improved flavor profiles relative to prior-art formulations. Without limiting this disclosure or any patent claim that matures from this disclosure, compositions comprising kavalactones formulated with other aromatic compounds allow for non-covalent interactions such as pi-stacking interactions between the kavalactones and the aromatic compounds, which non-covalent interactions compete with interactions with taste receptors and therefore inhibit the perception of the unfavorable flavors of kavalactones. Suitable aromatics include, without limitation, anthocyanins, glycosides of anthocyanins, flavonoids, and glycosides of flavonoids as well as other aromatic compounds described in the detailed description that follows.

Various aspects of this disclosure relate to the discovery that compositions comprising kavalactones formulated with cinnamaldehyde display improved pharmacokinetics relative to prior-art formulations. Without limiting this disclosure or any patent claim that matures from this disclosure, compositions comprising kavalactones formulated with cinnamaldehyde allow for non-covalent interactions such as pi-stacking interactions between the kavalactones and the cinnamaldehyde, which inhibits aggregation of the kavalactones and/or allows dispersal of the kavalactones in a composition, which inhibition of aggregation and/or dispersal increases the probability that the kavalactones will contact and be absorbed by the epithelial lining of the gastrointestinal tract between the lips and the stomach and therefore improves pharmacokinetics (e.g., as described above).

Various aspects of this disclosure relate to the discovery that compositions comprising kavalactones formulated with cinnamaldehyde display improved bioavailability relative to prior-art formulations. Without limiting this disclosure or any patent claim that matures from this disclosure, compositions comprising kavalactones formulated with cinnamaldehyde allow for non-covalent interactions such as pi-stacking interactions between the kavalactones and the cinnamaldehyde, which inhibits aggregation of the kavalactones and/or allows dispersal of the kavalactones in a composition, which inhibition of aggregation and/or dispersal increases the probability that the kavalactones will contact and be absorbed by the epithelial lining of the gastrointestinal tract between the lips and the stomach and therefore improves bioavailability (e.g., as described above).

Various aspects of this disclosure relate to the discovery that compositions comprising kavalactones formulated with cinnamaldehyde display attenuated side effects relative to prior-art formulations. Without limiting this disclosure or any patent claim that matures from this disclosure, compositions comprising kavalactones formulated with cinnamaldehyde allow for non-covalent interactions such as pi-stacking interactions between the kavalactones and the cinnamaldehyde, which inhibits aggregation of the kavalactones and/or allows dispersal of the kavalactones in a composition, which inhibition of aggregation and/or dispersal increases the probability that the kavalactones will contact and be absorbed by the epithelial lining of the gastrointestinal tract between the lips and the stomach and therefore attenuates side effects (e.g., as described above).

Various aspects of this disclosure relate to the discovery that compositions comprising kavalactones formulated with cinnamaldehyde display improved flavor profiles relative to prior-art formulations. Without limiting this disclosure or any patent claim that matures from this disclosure, compositions comprising kavalactones formulated with cinnamaldehyde allow for non-covalent interactions such as pi-stacking interactions between the kavalactones and the cinnamaldehyde, which non-covalent interactions compete with interactions with taste receptors and therefore inhibit the perception of the unfavorable flavors of kavalactones.

Various aspects of this disclosure relate to the discovery that (1) one or more alcohols as a solvent and/or cosolvent, (2) oral sprays, (3) aromatic compounds, and/or (4) cinnamaldehyde display a synergistic effect on the pharmacokinetics of kavalactones, for example, because one or more described above are super-additive.

Various aspects of this disclosure relate to the discovery that (1) one or more alcohols as a solvent and/or cosolvent, (2) oral sprays, (3) aromatic compounds, and/or (4) cinnamaldehyde display a synergistic effect on the bioavailability of kavalactones, for example, because one or more effects described above are super-additive.

Various aspects of this disclosure relate to the discovery that (1) one or more alcohols as a solvent and/or cosolvent, (2) oral sprays, (3) aromatic compounds, and/or (4) cinnamaldehyde display a synergistic effect on the inhibition of the side effects of kavalactones, for example, because one or more of the effects described above are super-additive.

Various aspects of this disclosure relate to the discovery that aromatic compounds and cinnamaldehyde display a synergistic effect on masking the flavor of kavalactones, for example, because one or more of the effects described above are super-additive.

The foregoing advantages and other advantages of the compositions of this disclosure will become immediately apparent in view of the foregoing and the following detailed description. Nothing in this summary section or the following detailed description shall limit any patent claim that matures from this disclosure, which patent claim(s) shall instead be construed according to the plain meaning of the language used in the patent claim(s) in the context of each claim's dependency, and, if any ambiguity remains, in accordance with recognized canons of claim construction.

### DETAILED DESCRIPTION

Various aspects of this disclosure relate to a composition comprising one or more kavalactones. In some embodiments, the one or more kavalactones are selected from kavain, 5-hydroxykavain, 7,8-dihydrokavain, 11-methoxy-12-hydroxydehydrokavain, methysticin, 1,8-dihydromethysticin, 5,6-dehydromethysticin, 7,8-dihydromethysticin, yangonin, desmethoxyyangonin, 11-hydroxyyangonin, 10-methoxyyangonin, 11-methoxyyangonin, 5,6-dihydroyangonin, 7,8-dihydroyangonin, 5,6,7,8-tetrahydroyangonin, and 11-methoxy-12-hydroxydehydrokavain. In some specific embodiments, the one or more kavalactones are selected from 1,8-dihydromethysticin, 7,8-dihydrokavain, desmethoxyyangonin, kavain, methysticin, and yangonin. In some very specific embodiments, the one or more kavalactones consist of kavain.

In some embodiments, the composition comprises one or more aromatic compounds. In some specific embodiments, the one or more aromatic compounds are selected from apocynin, albaspidin, albofungin, alpha-thujaplicin, alpha-zearalenol, para-anol, anthralin, anthranol, anthrarobin, arbutamine, ascofuranone, aspidin, aspidinol, atranorin, bakuchiol, benzarone, benzbromarone, benziodarone, beta-thujaplicin, beta-zearalenol, butopyronoxyl, calphostin A, calphostin B, calphostin C, calphostin D, cavicularin, chaetochromin A, chaetochromin B, chaetochromin C, chaetochromin D, chlorindanol, chrysarobin, clofoctol, clorophene, collinomycin, coniferyl alcohol, ortho-cresol, meta-cresol, para-cresol, curvularin, danthron, 4,6-di-tert-butyl-meta-cresol, deferiprone, desaspidin BB, dianol, dioxybenzone, diresorcinol, dobutamine, drupanol, durantin A, embelin, enterolactone, epicocconone, ethyl maltol, fenoterol, fenretinide, flopropione, fumigatin, fusarubin, gallein, gamma-thujaplicin, gentisyl alcohol, gossypol, herqueinone, homosalate, humulone, hydrocarbostyril, 9-hydroxychrysarobin, hydroxytyrosol, isohumulone, isoliquiritigenin, isomaltol, ivacaftor, javanicin, alpha-kosin, beta-kosin, kuwanon G, laquinimod, leptosphaerin A, leptosphaerin B, linomide, lupulon, maltol, menadiol, mequinol, monobenzone, morphenol, myricanone, nimbiol, nordihydroguaiaretic acid, orthocaine, osalmid, oxyphenbutazone, paroxypropione, perezone, phenolphthalol, probucol, procerin, propyl gallate, protiofate, protokosin, psiguadial A, raspberry ketone, rottlerin, rottlerin 5,7-dimethyl ether, sappanol, scillavone A, scillavone B, selligueain A, geranin A, sesamol, siccanin, sophoraflavanone G, sparassol, stigmatellin, sumatrol, taleranol, uliginosin A, uliginosin B, urolithin A, urolithin B, vanillin, vanillyl alcohol, verrulactone A, verrulactone B, verrulactone E, violacein, viridicatin, xanthoxylin, zearalenone, zeranol, resistomycin, amylmetacresol, chavicol, 4-hydroxyphenylacetaldehyde, oleocanthal, ortho-benzylphenol, ortho-phenylphenol, para-benzyphenol, propofol, protocatechualdehyde, pseudoisoeugenol, salicyl alcohol, salicylaldehyde, salicylanilide, para-tert-pentyl-phenol, tyrosol, xibornol, apocynin, chavibetol, DOPAL, DOPEG, eugenol, [6]-gingerol, guaiacol, homovanillyl alcohol, hydroxytyrosol, isoeugenol, 3-methoxy-4-hydroxyphenylglycol, para-vinylguaiacol, shogaol, strobilurin F, zingerone, adipostatin A, beta-resorcylaldehyde, bilobol, 4-hexylresorcinol, olivetol, orcinol, resorcinol, acetosyringone, canolol, purpurogallin, syringaldehyde, syringol, butylparaben, ethylparaben, heptylparaben, methylparaben, propylparaben, alpha-naphtholphthalein, naphthoresorcinol, 2-naphthol, phylloquinol, spinochrome B, ubiquinol, 2,5-di-tert-pentyl-hydroquinone, geranylhydroquinone, ilimaquinone, alkannin, atovaquone, buparvaquone, juglone, lapachol, lawsone, naphthazarin, nigrosporin B, parvaquone, phthiocol, plumbagin, xylindein, alizarin, alizarin 1-methyl ether, alizarin 2-methyl ether, aloe emodin, anthragallol, anthrapurpurin, anthrarufin, damnacanthal, dantron, purpuroxanthin, quinizarin, oxyanthrarufin, flavopurpurin, oxychrysazin, 1,6-dihydroxyanthraquinone, emodin, parietin, purpurin, purpurin 2,4-dimethyl ether, purpurin 2-methyl ether, quinalizarin, rubiadin, 3-hydroxychrysazin, thunberginol F, leucocyanidin, leucofisetinidin, leucopelargonidin, leucopeonidin, melacacidin, silibinin A, silibinin B, euxanthone, gentisin, isojacareubin, mangostin, secalonic acid A, secalonic acid B, secalonic acid C, secalonic acid D, ergoflavin, ergochrysin A, euparin, usnic acid, sulfuretin, capsaicin, dihydrocapsaicin, homocapsaicin I, homocapsaicin II, homodihydrocapsaicin, nonivamide, nordihydrocapsaicin, butylated hydroxyanisole, butylated hydroxytoluene, alpha-peltatin, beta-peltatin, arctigenin, balanophonin, enterodiol, enterolactone, honokiol, hydroxymatairesinol, lariciresinol, macelignan, magnolol, matairesinol, 4-O-methylhonokiol, obovatol, pinoresinol, secoisolariciresinol, syringaresinol, resacetophenone, tolcapone, benzoresorcinol, cotoin, alpha-tocopherol, beta-tocopherol, eta-tocopherol, gamma-tocopherol, zeta2-tocopherol, delta-tocopherol, alpha-tocotrienol, beta-tocotrienol, gamma-tocotrienol, delta-tocotrienol, bisdemethoxycurcumin, 1,7-bis(4-hydroxyphenyl)-1,4,6-heptatrien-3-one, curcumin, cyclovalone, demethoxycurcumin, piceatannol, pinosylvin, pinosylvin monomethyl ether, pterostilbene, resveratrol, dihydroresveratrol, combretastatin, combretastatin A-1, combretastatin A-4, combretastatin B-1, 6-hydroxyluteolin, acacetin, acerosin, alnetin, amentoflavone, apigenin, artocarpetin, baicalein, cannflavin A, cannflavin B, cannflavin C, chrysin, chrysoeriol, cirsilineol, cirsiliol, cirsimaritin, corymbosin, datiscetin, 4′,7-dihydroxyflavone, 7,8-dihydroxyflavone, diosmetin, echioidinin, eupatilin, eupatorin, gardenin A, gardenin B, gardenin C, gardenin D, gardenin E, genkwanin, geraldone, hispidulin, 6-hydroxyflavone, hymenoxin, hypolaetin, isoscutellarein, isoxanthohumol, jaceosidin, luteolin, methylchrysin, mikanin, negletein, nepetin, nevadensin, nodifloretin, norartocarpetin, norwogonin, onopordin, oroxylin A, pectolinarigenin, pedalitin, pilloin, pratol, primetin, primuletin, scaposin, scutellarein, serpyllin, sorbifolin, sudachitin, techtochrysin, tithonine, tricetin, tricin, velutin, wightin, wogonin, xanthomicrol, zapotinin, baptigenin, biochanin A, calycosin, derrubone, formononetin, genistein, glycitein, irilone, irigenin, irisolone, 5-O-methylgenistein, orobol, osajin, pomiferin, pratensein, prunetin, pseudobaptigenin, psi-tectorigenin, retusin, tectorigenin, wighteone, afzelechin, epiafzelechin, apiforol, catechin, epicatechin, epigallocatechin, epicatechin gallate, epigallocatechin gallate, fisetinidol, gallocatechol, guibourtinidol, isosilybin A, isosilybin B, isosilychristin, luteoforol, meciadanol, mesquitol, oritin, ourateacatechin, robinetinidol, silybin A, silybin B, silychristin, silydianin, aromadendrin, axillarin, azaleatin, dihydrokaempferide, ermanin, fisetin, galangin, gossypetin, kaempferide, kaempferol, isorhamnetin, laricitrin, mearnsetin, morin, myricetin, pachypodol, pinobanksin, quercetagetin, quercetin, rhamnazin, rhamnetin, spinacetin, syringetin, taxifolin, ampelopsin, aromadedrin, blumeatin, butin, dihydrogossypetin, dihydromorin, eriodictyol, fustin, garbanzol, hesperetin, homoeriodictyol, isosakuranetin, liquiritigenin, naringenin, pinocembrin, pinostrobin, poriol, sakuranetin, sterubin, dehydroequol, equol, glabridin, lonchocarpane, laxiflorane, luteone, glabrene, aureusidin, leptosidin, oryzanol A, oryzanol C, campesteryl ferulate, sitosteryl ferulate, stigmasteryl ferulate, sitostanyl ferulate, glyceollin I, glyceollin II, glyceollin III, glyceollin IV, glycinol, glycyrrhizol, medicarpin, phaseolin, cabenegrin A-I, cabenegrin A-II, dihydrokanakugiol, kanakugiol, licochalcone A, licochalcone B, licochalcone C, licochalcone D, licochalcone E, phloretin, sophoradin, xanthohumol, plicadin, psoralidin, wedelolactone, carvacrol, ferruginol, mutisianthol, taxodione, taxodone, thymol, and one or more glycosides of one or more of the foregoing. In some embodiments, the one or more aromatic compounds are selected from anthocyanins, glycosides of anthocyanins, flavonoids, and glycosides of flavonoids. In some specific embodiments, the one or more aromatic compounds are selected from cyanidin, cyanidin 3-rutinoside, cyanidin 3-glucoside, cyanidin 3-glucosylrutinoside, cyanidin 3-sophoroside, pelargonidin, and peonidin 3-glucoside. In some very specific embodiments, the one or more aromatic compounds are selected from cyanidin 3-rutinoside and cyanidin 3-glucosylrutinoside, which are glycosylated anthocyanins present in tart cherry.

In some embodiments, the composition comprises the one or more aromatic compounds and the one or more kavalactones at a molar ratio of at least 1:2. In some specific embodiments, the composition comprises the one or more aromatic compounds and the one or more kavalactones at a molar ratio of at least 1:1. In some very specific embodiments, the composition comprises the one or more aromatic compounds and the one or more kavalactones at a molar ratio of at least 1:1 and no greater than 5:1.

In some embodiments, the composition comprises cinnamaldehyde. In some specific embodiments, the composition comprises cinnamaldehyde and styrene. In some specific embodiments, the composition comprises cinnamaldehyde and one or both of cinnamic acid and ethyl cinnamate. In some very specific embodiments, the composition comprises cinnamaldehyde, styrene, cinnamic acid, and ethyl cinnamate.

In some embodiments, the composition comprises the cinnamaldehyde and the one or more kavalactones at a molar ratio of at least 1:5. In some specific embodiments, the composition comprises the cinnamaldehyde and the one or more kavalactones at a molar ratio of at least 1:2. In some very specific embodiments, the composition comprises the cinnamaldehyde and the one or more kavalactones at a molar ratio of at least 1:2 and no greater than 4:1.

In some embodiments, the composition comprises stevia extract. In some specific embodiments, the composition comprises stevia extract, and the stevia extract comprises steviol glycosides. In some very specific embodiments, the composition comprises stevia extract, the stevia extract comprises steviol glycosides, and the steviol glycosides comprise one or both of stevioside and rebaudioside A.

In some embodiments, the composition comprises a solvent. In some specific embodiments, the composition comprises a solvent, and the one or more kavalactones are dissolved in the solvent. In some very specific embodiments, the composition comprises a solvent, the one or more kavalactones are dissolved in the solvent, and the one or more aromatic compounds are dissolved in the solvent. In some very specific embodiments, the composition comprises a solvent, the one or more kavalactones are dissolved in the solvent, and the cinnamaldehyde is dissolved in the solvent. The term “solvent” as used in this disclosure refers to the most prevalent molecule in a composition by mass.

In some embodiments, the solvent is selected from water, ethanol, propylene glycol, and glycerol. In some specific embodiments, the solvent is water. In some specific embodiments, the solvent is ethanol. In some specific embodiments, the solvent is propylene glycol. In some specific embodiments, the solvent is glycerol.

In some embodiments, the one or more kavalactones are dissolved in the solvent at a concentration of at least 0.1 percent by mass. In some specific embodiments, the one or more kavalactones are dissolved in the solvent at a concentration of at least 0.5 percent by mass. In some very specific embodiments, the one or more kavalactones are dissolved in the solvent at a concentration of at least 0.5 percent and no greater than 20 percent by mass.

In some embodiments, the composition has a concentration of the one or more kavalactones that are dissolved in the solvent, the one or more kavalactones have a solubility in water at 98.6 degrees Fahrenheit, and the concentration of the one or more kavalactones that are dissolved in the solvent is greater than the solubility of the one or more kavalactones in water at 98.6 degrees Fahrenheit. Without limiting this disclosure or any patent claim that matures from this disclosure, a composition comprising kavalactones at a greater concentration than the solubility of the kavalactones in water at 98.6 degrees Fahrenheit favors the partitioning of the kavalactones out of the composition and into the epithelial lining of the mouth and esophagus following consumption of the composition as water within the mouth and/or lumen of the esophagus combines with the composition to dilute the solvent and any cosolvent(s), which solvent and any cosolvent(s) allow for the increased solubility of the one or more kavalactones in the composition relative to the solubility of the one or more kavalactones in water.

In some embodiments, the one or more aromatic compounds are dissolved in the solvent at a concentration of at least 0.1 percent by mass. In some specific embodiments, the one or more aromatic compounds are dissolved in the solvent at a concentration of at least 0.5 percent by mass. In some very specific embodiments, the one or more aromatic compounds are dissolved in the solvent at a concentration of at least 0.5 percent and no greater than 20 percent by mass.

In some embodiments, the cinnamaldehyde is dissolved in the solvent at a concentration of at least 0.02 percent by mass. In some specific embodiments, the cinnamaldehyde is dissolved in the solvent at a concentration of at least 0.05 percent by mass. In some very specific embodiments, the cinnamaldehyde is dissolved in the solvent at a concentration of at least 0.05 percent and no greater than 5 percent by mass.

In some embodiments, the one or more steviol glycosides are dissolved in the solvent at a concentration of at least 0.05 percent by mass. In some specific embodiments, the one or more steviol glycosides are dissolved in the solvent at a concentration of at least 0.1 percent by mass. In some very specific embodiments, the one or more steviol glycosides are dissolved in the solvent at a concentration of at least 0.1 percent and no greater than 10 percent by mass.

In some embodiments, the composition comprises a cosolvent. In some specific embodiments, the cosolvent is selected from water, ethanol, propylene glycol, and glycerol. In some very specific embodiments, the cosolvent is water. In some very specific embodiments, the cosolvent is ethanol. In some very specific embodiments, the cosolvent is propylene glycol. In some very specific embodiments, the cosolvent is glycerol. The term “cosolvent” as used in this disclosure refers to the second-most-prevalent molecule in a composition by mass.

In some embodiments, the solvent and the cosolvent are selected from ethanol, propylene glycol, glycerol, and water.

In some embodiments, the solvent is glycerol, and the cosolvent is ethanol.

In some embodiments, the solvent is glycerol, and the cosolvent is propylene glycol.

In some embodiments, the solvent is glycerol, and the cosolvent is water.

In some embodiments, the solvent is propylene glycol, and the cosolvent is ethanol.

In some embodiments, the solvent is propylene glycol, and the cosolvent is glycerol.

In some embodiments, the solvent is propylene glycol, and the cosolvent is water.

In some embodiments, the solvent is ethanol, and the cosolvent is glycerol.

In some embodiments, the solvent is ethanol, and the cosolvent is propylene glycol.

In some embodiments, the solvent is ethanol, and the cosolvent is water.

In some embodiments, the solvent is water, and the cosolvent is ethanol.

In some embodiments, the solvent is water, and the cosolvent is propylene glycol.

In some embodiments, the solvent is water, and the cosolvent is glycerol.

In some embodiments, the composition comprises ethanol, propylene glycol, and glycerol.

In some embodiments, the composition comprises ethanol, propylene glycol, and water.

In some embodiments, the composition comprises ethanol, glycerol, and water.

In some embodiments, the composition comprises propylene glycol, glycerol, and water.

In some embodiments, the composition comprises ethanol, propylene glycol, glycerol, and water.

In some embodiments, the composition comprises glycerol at a concentration of at least 10 percent by mass. In some specific embodiments, the composition comprises glycerol at a concentration of at least 10 percent and no greater than 75 percent by mass. In some very specific embodiments, the composition comprises glycerol at a concentration of at least 10 percent and no greater than 55 percent by mass.

In some embodiments, the composition comprises ethanol at a concentration of at least 10 percent by mass. In some specific embodiments, the composition comprises ethanol at a concentration of at least 10 percent and no greater than 55 percent by mass. In some very specific embodiments, the composition comprises ethanol at a concentration of at least 10 percent and no greater than 35 percent by mass.

In some embodiments, the composition comprises propylene glycol at a concentration of at least 5 percent by mass. In some specific embodiments, the composition comprises propylene glycol at a concentration of at least 5 percent and no greater than 45 percent by mass. In some very specific embodiments, the composition comprises propylene glycol at a concentration of at least 5 percent and no greater than 25 percent by mass.

In some embodiments, the composition comprises water at a concentration of at least 5 percent by mass. In some specific embodiments, the composition comprises water at a concentration of at least 5 percent and no greater than 45 percent by mass. In some very specific embodiments, the composition comprises water at a concentration of at least 5 percent and no greater than 25 percent by mass.

In some embodiments, the composition is a liquid. In some specific embodiments, the composition is a liquid, and the composition comprises at least 100 milligrams and no greater than 5 grams of the one or more kavalactones. Such liquid compositions include, for example, compositions that consist of 5 to 50 milliliters contained within a tincture bottle or spray bottle for retail sale.

In some embodiments, the composition is an aerosol. The composition may be formed, for example, by spraying a liquid composition according to this disclosure to produce an aerosol. In some specific embodiments, the composition is an aerosol, and the composition comprises at least 1 milligram and no greater than 100 milligrams of the one or more kavalactones. In some very specific embodiments, the composition is an aerosol, and the composition comprises at least 1 milligram and no greater than 20 milligrams of the one or more kavalactones.

Various aspects of this disclosure relate to a container that contains a composition described anywhere in this disclosure. In some embodiments, the container is hermetically sealed. In some embodiments, the container comprises a sprayer. In some very specific embodiments, the container comprises a sprayer that is configured to spray a liquid composition according to this disclosure to form an aerosol composition according to this disclosure.

In some embodiments, the container is configured to spray at least 20 milliliters and no greater than 200 milliliters of the composition per spray. In some specific embodiments, the container is configured to spray at least 50 milliliters and no greater than 150 milliliters of the composition per spray. In some very specific embodiments, the container is configured to spray at least 80 milliliters and no greater than 120 milliliters of the composition per spray.

Various aspects of this disclosure relate to a method to consume kavalactones, comprising spraying a liquid composition according to this disclosure (e.g., from a container of the preceding paragraph) into a mouth of a person to form an aerosol composition according to this disclosure in the mouth of the person.

In some embodiments, a composition of this disclosure has an oral Tmax of less than 60 minutes for the one or more kavalactones, wherein the oral Tmax is the time it takes to achieve a maximum plasma concentration of the one or more kavalactones in the person after spraying the composition into the mouth of the person. In some specific embodiments, a composition of this disclosure has an oral Tmax of less than 45 minutes. In some very specific embodiments, a composition of this disclosure has an oral Tmax of less than 15 minutes.

In some embodiments, a composition of this disclosure has a bioavailability of at least 10 percent for the one or more kavalactones (e.g., when consumed by spraying the composition into the mouth of a person). In some specific embodiments, a composition of this disclosure has a bioavailability of at least 20 percent for the one or more kavalactones. In some very specific embodiments, a composition of this disclosure has a bioavailability of at least 35 percent for the one or more kavalactones.

### EXEMPLIFICATION

Example 1. Ingredients of a liquid kavalactone composition.

A liquid composition is prepared comprising 5 to 15 percent by mass kava extract containing about 70 percent by mass kavalactones (resulting in about 3.5 to about 10 percent by mass of the kavalactones in the liquid composition). The kavalactones comprise kavain, methysticin, yangonin, 7,8-dihydrokavain, 1,8-dihydromethysticin, and desmethoxyyangonin in order from most abundant (kavain) to least abundant (desmethoxyyangonin). The majority of the kavalactones consist of kavain, methysticin, and yangonin by mass. The kava extract is dissolved in 20 to 35 percent by mass glycerol, 15 to 30 percent by mass ethanol, 5 to 20 percent by mass propylene glycol, and 2 to 15 percent by mass water. Tart cherry extract is also added to the liquid composition at a concentration of 5 to 15 percent by mass, wherein the tart cherry extract comprises about 50 percent aromatic compounds according to this disclosure. The aromatic compounds consist primarily of glycosides of anthocyanidins including cyanidin 3-glucosylrutinoside (most prevalent), cyanidin 3-rutinoside (second-most prevalent), and lower concentrations of peonidin-3-glucoside, cyanidin 3-sophoroside, cyanidin 3-glucoside, cyanidin, and pelargonidin in order from most abundant (peonidin-3-glucoside) to least abundant (pelargonidin). Cinnamon flavor is also added to the liquid composition at a concentration of 0.2 to 4 percent by mass, wherein the cinnamon flavor comprises cinnamaldehyde at a concentration of about 70 percent and lesser concentrations of styrene, cinnamic acid, and ethyl cinnamate. Stevia extract is also added to the liquid composition at a concentration of 0.5 to 5 percent by mass, wherein the stevia extract comprises steviol glycosides including stevioside and rebaudioside A.

Example 2. A container comprising a liquid kavalactone composition.

A container comprising a sprayer is loaded with 10 milliliters of a liquid kavalactone composition according to Example 1 and hermetically sealed to inhibit atmospheric oxygen from entering the container and oxidizing the liquid composition. The sprayer is configured to spray about 80 to 120 microliters of the liquid composition to produce an aerosol.

Example 3. A method to consume a kavalactone composition.

The container of Example 2 is used to spray the liquid composition of Example 1 to produce an aerosol composition comprising about 80 to 120 microliters of the liquid composition and about 3 milligrams to 12 milligrams of the kavalactones. The aerosol composition is sprayed into the mouth of a person. The blood plasma of the person is monitored to determine the Tmax of the kavalactones, which is less than 45 minutes, and to determine that the bioavailability of the kavalactones is greater than 20 percent. The person experiences psychoactive effects associated with the kavalactones within about 10 minutes of consuming the spray.

Nothing in the preceding specification and exemplification shall limit any patent claim that matures from this disclosure, which patent claim(s) shall instead be construed according to the plain meaning of the language used in the patent claim(s) in the context of each claim's dependency, and, if any ambiguity remains, in accordance with recognized canons of claim construction.

What is claimed is:

1. A composition, comprising one or more kavalactones, one or more aromatic compounds, and a solvent, wherein:
the one or more kavalactones are selected from 1,8-dihydromethysticin, 7,8-dihydrokavain, desmethoxyyangonin, kavain, methysticin, and yangonin;
the one or more aromatic compounds are selected from anthocyanins, glycosides of anthocyanins, flavonoids, and glycosides of flavonoids;
the one or more kavalactones are dissolved in the solvent at a concentration of at least 0.5 percent by mass; and
the one or more aromatic compounds are dissolved in the solvent at a concentration of at least 0.5 percent by mass.

2. The composition of claim 1, comprising the one or more aromatic compounds and the one or more kavalactones at a molar ratio of at least 1:1.

3. The composition of claim 1, wherein:
the composition has a concentration of the one or more kavalactones that are dissolved in the solvent;
the one or more kavalactones have a solubility in water at 98.6 degrees Fahrenheit; and
the concentration of the one or more kavalactones that are dissolved in the solvent is greater than the solubility of the one or more kavalactones in water at 98.6 degrees Fahrenheit.

4. The composition of claim 1, wherein the composition is a liquid.

5. The composition of claim 1, wherein the composition is an aerosol.

6. The composition of claim 5, comprising at least 1 milligram and no greater than 100 milligrams of the one or more kavalactones.

7. The composition of claim 1, comprising a cosolvent, wherein the solvent and the cosolvent are selected from ethanol, propylene glycol, glycerol, and water.

8. The composition of claim 7, comprising each of ethanol, propylene glycol, glycerol, and water.

9. The composition of claim 7, comprising glycerol at a concentration of at least 10 percent by mass.

10. The composition of claim 7, comprising ethanol at a concentration of at least 10 percent by mass.

11. The composition of claim 7, comprising propylene glycol at a concentration of at least 5 percent by mass.

12. The composition of claim 7, comprising water at a concentration of at least 5 percent by mass.

13. The composition of claim 1, comprising cinnamaldehyde at a concentration of at least 0.05 percent by mass.

14. The composition of claim 1, wherein the one or more aromatic compounds are selected from cyanidin, cyanidin 3-rutinoside, cyanidin 3-glucoside, cyanidin 3-glucosylrutinoside, cyanidin 3-sophoroside, pelargonidin, and peonidin-3-glucoside.

15. The composition of claim 1, wherein the one or more aromatic compounds comprise cyanidin 3-glucosylrutinoside.

16. The composition of claim 1, wherein the one or more aromatic compounds comprise cyanidin 3-rutinoside.

17. A container, comprising a sprayer and containing a composition, wherein the composition comprises one or more kavalactones.

18. A method to consume kavalactones, comprising spraying a composition comprising one or more kavalactones from a container into a mouth of a person, wherein the container comprises a sprayer.

19. The method of claim 18, wherein:
the composition has an oral Tmax of less than 45 minutes for the one or more kavalactones; and
the oral Tmax is the time it takes to achieve a maximum plasma concentration of the one or more kavalactones in the person following the spraying.

20. The method of claim 18, wherein the one or more kavalactones display a bioavailability of at least 20 percent in the person.
